(19) United States
(12) Patent Application Publication
(10) Pub. No.: US 2025/0067745 A1
(43) Pub. Date: Feb. 27, 2025

(54) CD38 AS A BIOMARKER AND BIOTARGET IN T-CELL LYMPHOMAS

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); UNIVERSITÉ PARIS CITÉ, Paris (FR)

(72) Inventors: Armand BENSUSSAN, Paris (FR); Maxime BATTISTELLA, Paris Cedex 10 (FR); Adèle DE MASSON, Paris (FR); Martine BAGOT, Paris (FR); Hélène MOINS, Paris (FR)

(21) Appl. No.: 18/725,312

(22) PCT Filed: Jan. 27, 2023
(86) PCT No.: PCT/EP2023/052000

§ 371 Date: Jun. 28, 2024

(30) Foreign Application Priority Data
Jan. 31, 2022 (EP) 22305106.1

(51) Int. Cl. G01N 33/574; C12Q 1/6874; C12Q 1/6886

(52) U.S. Cl. CPC G01N 33/57492; C12Q 1/6874; C12Q 1/6886; G01N 33/57407; C12Q 2600/118; C12Q 2600/158

(57) ABSTRACT

T-cell lymphomas are a heterogeneous group of malignancies involving T lymphocytes and generally characterized by a poor prognosis. Among them, cutaneous T-cell lymphomas involve primarily the skin. Mycosis fungoides and Sézary syndrome are the most frequent cutaneous T-cell lymphomas. Now the inventors showed the expression of CD38 by Sézary cells and in CD4+ blood cells of patients with Sezary syndrome. CD38 therefore appears as a useful diagnostic, prognostic and follow-up marker, and as a potential therapeutic target in T-cell lymphomas. Therapeutic depletion of CD38-expressing cancer cells would eliminate tumor cells.

### FIELD OF THE INVENTION

The present invention is in the field of medicine, in particular oncology.

### BACKGROUND OF THE INVENTION

T-cell lymphomas are a heterogeneous group of malignancies involving T lymphocytes and generally characterized by a poor prognosis. Among them, cutaneous T-cell lymphomas involve primarily the skin. Mycosis fungoides and Sézary syndrome are the most frequent cutaneous T-cell lymphomas. Sézary syndrome is defined as erythroderma (erythema of the entire skin surface), and circulating tumor blood cells (1). The circulating tumor T cells express CD4 and may lose expression of CD7 and CD26, while exhibiting in most cases aberrant expression of CD158k (KIR3DL2), which is a surface marker of tumor T cells in Sézary syndrome (2). The initial diagnosis of the disease is difficult and the monitoring of blood involvement is complicated because international criteria use the loss of CD7 and CD26 markers (CD4+CD26− and CD4+CD7− cells) (3) which is not specific for tumor cells (4). The discovery by Martine Bagot and Armand Bensussan of CD158k (KIR3DL2) expression by Sézary cells, allowed the use of the KIR3DL2 marker for the diagnosis, disease monitoring (2) and the development of a therapeutic monoclonal antibody (lacutamab). Lacutamab has been tested in a phase I study with published results (5) and is currently being studied in cutaneous T-cell lymphomas and other peripheral T-cell lymphomas in a phase II international multicenter prospective trial. However, long-term responses are rare and new treatments are needed. Recently, treatment with anti-CCR4 monoclonal antibody (mogamulizumab) has improved progression-free survival in cutaneous T-cell lymphomas (6). However, CCR4 is expressed not only by Sézary cells but also by memory regulatory T cells of the peripheral blood, and its use is associated with the occurrence of autoimmune adverse reactions (7). Besides CCR4, Sézary cell expresses several markers common with regulatory T lymphocytes, such as PD1 (8), CD39 (9) and TIGIT (10). Thus there is a need for identifying new markers and targets to the treatment of T-cell lymphomas.

### SUMMARY OF THE INVENTION

The present invention is defined by the claims. In particular, the present invention relates to methods for the diagnosis and treatment of T-cell lymphomas.

### DETAILED DESCRIPTION OF THE INVENTION

The inventors showed the expression of CD38 by Sézary cells and in CD4+ blood cells of patients with Sézary syndrome. CD38 therefore appears as a useful diagnostic, prognostic and follow-up marker, and as a potential therapeutic target in T-cell lymphomas. Therapeutic depletion of CD38-expressing cancer cells would eliminate tumor cells.

### Main Definitions

As used herein, the term “T cell” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8+ T cells, CD4+ T cells, and gamma delta T cells. As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor infiltrating CD8+ T cell” refers to the pool of CD8+ T cells of the patient that have left the blood stream and have migrated into a tumor. As used herein, the term “CD4+ T cells” (also called T helper cells or TH cells) refers to T cells which express the CD4 glycoprotein on their surfaces and which assist other white blood cells in immunologic processes, including maturation of B cells into plasma cells and memory B cells, and activation of cytotoxic T cells and macrophages. CD4+ T cells become activated when they are presented with peptide antigens by MHC class II molecules, which are expressed on the surface of antigen-presenting cells (APCs). Once activated, they divide rapidly and secrete cytokines that regulate or assist in the active immune response. These cells can differentiate into one of several subtypes, including TH1, TH2, TH3, TH17, TH9, TFH or Treg, which secrete different cytokines to facilitate different types of immune responses. Signaling from the APC directs T cells into particular subtypes. In addition to CD4, the TH cell surface biomarkers known in the art include CXCR3 (Th1), CCR4, Crth2 (Th2), CCR6 (Th17), CXCR5 (Tfh) and as well as subtype-specific expression of cytokines and transcription factors including T-bet, GATA3, EOMES, RORγT, BCL6 and FoxP3. As used herein, the term “gamma delta T cell” has its general meaning in the art. Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MHC molecules of antigen-presenting cells. Gamma 9 delta 2 T cells (sometimes also called gamma 2 delta 2 T cells) are gamma delta T cells bearing TCR receptors with the variable domains Vγ9 and Vδ2. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62: 1-5) or by other intracellular parasites, such as mycobacteria (Constant et al., Infection and Immunity, December 1995, vol. 63, no. 12: 4628-4633) or protozoa (Behr et al., Infection and Immunity, 1996, vol. 64, no. 8: 2892-2896). They may also be cancer cells (Poccia et al., J. Immunol., 159: 6009-6015; Fournie and Bonneville, Res. Immunol., 66th Forum in Immunology, 147: 338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

As used herein, the term “T-cell lymphoma” has its general meaning in the art and refers to a rare form of cancerous lymphoma affecting T-cells. Lymphoma arises mainly from the uncontrolled proliferation of T-cells and can become cancerous. T-cell lymphoma is categorized under Non-Hodgkin Lymphoma (NHL) and represents less than 15% of all Non-Hodgkin's diseases in the category. T-cell lymphomas are often categorised based on their growth patterns as either; aggressive (fast-growing) or indolent (slow-growing). In particular, T-cell lymphomas include peripheral T-cell lymphomas, Angioimmunoblastic T-cell lymphoma (AITL), and Cutaneous T-cell lymphoma (CTCL). In some embodiments, the T-cell lymphoma is gamma delta T-cell lymphoma, Hepatosplenic T-cell lymphoma (HSTCL), Angioimmunoblastic T-cell lymphoma (AITL), aggressive epidermotropic cutaneous T-cell lymphoma, primary cutaneous anaplastic large-cell lymphoma, NK/T-cell lymphoma (NKTL), Mycosis fungoide (MF) or Sézary syndrome (SS). In some embodiments, the T-cell lymphoma is a cutaneous T-cell lymphoma. In some embodiments, the cutaneous T-cell lymphoma is selected from the group consisting in Sézary Syndrome, Mycosis Fungoides, NK/T cell lymphoma, gamma delta T-cell lymphoma, aggressive epidermotropic cutaneous T-cell lymphoma or primary cutaneous anaplastic large-cell lymphoma.

As used herein, the term “cutaneous T-cell lymphoma” or “CTCL” has its general meaning in the art and refers to a rare heterogeneous group of non-Hodgkin lymphomas derived from skin-homing mature T-cells. Mycosis fungoides (MF) and Sézary Syndrome (SS) represent the most common subtypes of primary CTCL, with an incidence rate of 4.1/1,000,000 person-years and male predominance.

As used herein the term “Sézary syndrome” or “SS” has its general meaning in the art and refers to an aggressive form of cutaneous T-cell lymphoma characterized by a triad of erythroderma, lymphadenopathy and circulating atypical lymphocytes (Sézary cells). SS develops most frequently in men, is more frequent in the elderly, and progresses rapidly. SS correspond to stages IVA2 and IVB of T-cell cutaneous lymphoma (see this term). Patients present with a scaling erythroderma and infiltration often manifesting with leonine facies and severe pruritus. Alopecia, ectropium, mild palmoplantar keratoderma and nail onychodystrophy may be present. Lymphadenopathy and hepatosplenomegaly are observed. Patients often shiver and complain of chills and general fatigue.

As used herein, the term “CD38” has its general meaning in the art and refers to the ADP-ribosyl cyclase/cyclic ADP-ribose hydrolase 1. An exemplary amino acid sequence for CD38 is represented by SEQ ID NO:1. The extracellular domain of CD38 ranges from the amino acid residue at position 43 to the amino acid residue at position 300 in SEQ ID NO:1.

>sp|P28907|CD38_HUMAN ADP-ribosyl cyclase/cyclic ADP-ribose
hydrolase 1 OS = Homo sapiens OX = 9606 GN = CD38 PE = 1 SV = 2
SEQ ID NO: 1
MANCEFSPVSGDKPCCRLSRRAQLCLGVSILVLILVVVLAVVVPRWRQQWSGPGTTKRFP
ETVLARCVKYTEIHPEMRHVDCQSVWDAFKGAFISKHPCNITEEDYQPLMKLGTQTVPCN
KILLWSRIKDLAHQFTQVQRDMFTLEDTLLGYLADDLTWCGEFNTSKINYQSCPDWRKDC
SNNPVSVFWKTVSRRFAEAACDVVHVMLNGSRSKIFDKNSTFGSVEVHNLQPEKVQTLEA
WVIHGGREDSRDLCQDPTIKELESIISKRNIQFSCKNIYRPDKFLQCVKNPEDSSCTSEI

As used herein, the term “agent capable of inducing cell death of CD38 expressing cancer cells” refers to any molecule that under cellular and/or physiological conditions is capable of inducing cell death of CD38 expressing cancer cells. In particular, the agent is capable of inducing apoptosis of CD38 expressing cancer cells. In some embodiments, the agent is capable of depleting CD38 cancer cells.

As used herein, the term “depletion” with respect to cancer cells, refers to a measurable decrease in the number of CD38 expressing cancer cells in the patient. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of CD38 cancer cells in the patient below detectable limits.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

As used herein, the term “fully human” refers to an immunoglobulin, such as an antibody or antibody fragment, where the whole molecule is of human origin or consists of an amino acid sequence identical to a human form of the antibody or immunoglobulin.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

As used herein, the term “bispecific antibody” has its general meaning in the art and refers to an artificial, hybrid antibody having two different pairs of heavy and light chain and also two different antigen-binding sites.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. Moreover, when expressed in T-cells, CARs advantageously do not dimerize with endogenous T cell receptor (TCR) alpha and beta chains. The chimeric antigen receptor the present invention typically comprises an extracellular hinge domain, a transmembrane domain, and an intracellular T cell signaling domain.

As used herein the term “CAR-T cell” refers to a T lymphocyte that has been genetically engineered to express a CAR. The definition of CAR T-cells encompasses all classes and subclasses of T-lymphocytes including CD4+, CD8+ T cells, gamma delta T cells as well as effector T cells, memory T cells, regulatory T cells, and the like. The T lymphocytes that are genetically modified may be “derived” or “obtained” from the patient who will receive the treatment using the genetically modified T cells or they may “derived” or “obtained” from a different patient.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of drug are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of a drug of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a drug of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a drug of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a drug of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

### Methods of Treating:

Accordingly, the first object of the present invention relates to a method of treating a T-cell lymphoma in patient in need thereof comprising administering to the patient a therapeutically effective amount of an agent capable of inducing cell death of CD38 expressing cancer cells.

In some embodiments, the T-cell lymphoma is cutaneous T-cell lymphoma. More particularly, the T-cell lymphoma is Sézary syndrome.

In some embodiments, the patient is a human infant. In some embodiments, the patient is a human child. In some embodiments, the patient is a human adult. In some embodiments, the patient is an elderly human. In some embodiments, the patient is a premature human infant.

### CD38 Antibodies:

In some embodiments, the agent is an antibody having binding affinity for CD38. In some embodiments, the agent is an antibody directed against a least one extracellular domain of CD38. In some embodiments, the antibody leads to the depletion of CD38 expression cancer cells.

In some embodiments, the antibody is a humanized antibody or a chimeric antibody.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

Anti-CD38 antibodies are well known in the art (see Hashmi H, Husnain M, Khan A, Usmani S Z. CD38-Directed Therapies for Management of Multiple Myeloma. Immunotargets Ther. 2021 Jun. 29; 10:201-211. doi: 10.2147/ITT.S259122. PMID: 34235096; PMCID: PMC8254545).

In some embodiments, the anti-CD38 antibody is selected from the group consisting of isatuximab, daratumumab and felzartamab.

For instance, daratumumab has the heavy chain as set forth in SEQ ID NO:2 and the light chain as set forth in SEQ ID NO:3. The antibody binds a unique CD38 epitope at the C-terminal region of human CD38, amino acids 233 to 246 and 267 to 280, with amino acids in positions 272 and 274 being particularly important for binding.

>Daratumumab heavy chain
SEQ ID NO: 2
EVQLLESGGGLVQPGGSLRLSCAVSGFTFNSFAMSWVRQAPGKGLEWVSAISGSGGGTYY
ADSVKGRFTISRDNSKNTLYLQMNSLRAEDTAVYFCAKDKILWFGEPVFDYWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPELL
GGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
>Daratumumab light chain
SEQ ID NO: 3
EIVLTQSPATLSLSPGERATLSCRASQSVSSYLAWYQQKPGQAPRLLIYDASNRATGIPA
RFSGSGSGTDFTLTISSLEPEDFAVYYCQQRSNWPPTFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC

In some embodiments, the anti-CD38 antibody of the present invention comprises:
a heavy chain comprising i) the H-CDR1 as set forth in SEQ ID NO:4, ii) the H-CDR2 as set forth in SEQ ID NO:5 and iii) the H-CDR3 as set forth in SEQ ID NO:6, and,
a light chain comprising i) the L-CDR1 as set forth in SEQ ID NO:7, ii) the L-CDR2 as set forth in SEQ ID NO:8 and iii) the L-CDR3 as set forth in SEQ ID NO:9.

SEQ ID NO: 4 (H-CDR1):
GYTFTSYW
SEQ ID NO: 5 (H-CDR2):
IYPGDGDT
SEQ ID NO: 6 (H-CDR3):
ARERTTGAPRYFDV
SEQ ID NO: 7 (L-CDR1):
ENIYSF
SEQ ID NO: 8 (L-CDR2):
NTK
SEQ ID NO: 9 (L-CDR3):
QHHYGIPLT

In some embodiments, the monoclonal antibody of the present invention comprises a VH domain having at least 70% of identity with the amino acid sequence as set forth in SEQ ID NO:10.

In some embodiments, the monoclonal antibody of the present invention comprises a VL domain having at least 70% of identity with the amino acid sequence as set forth in SEQ ID NO:11.

>IgH VH1.87-D1.1-J1:
SEQ ID NO: 10
QVQLQQSGAELARPGASVKLSCKASGYTFTSYWMQWVKQRPGQGLEWIG
AIYPGDGDTRYTQKFKGKATLTADKSSSTAYMQLSNLTSEDSAVYYCAR
ERTTGAPRYFDVWGAGTTVTVSS
>Igk Vk12.44-Jk5:
SEQ ID NO: 11
DIQMTQSPASLSASVGETVTITCRASENIYSFLAWYQQKQGKSPQLLVY
NTKTLTEGVPSRFSGSGSGTQFSLKINNLQPEDFGSYYCQHHYGIPLTF
GAGTKLELK

According to the present invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence. According to the invention a first amino acid sequence having at least 90% of identity with a second amino acid sequence means that the first sequence has 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the anti-CD38 antibody of the present invention is a scFv fragment that consists of the amino acid sequence as set forth in SEQ ID NO:12.

>anti-CD38 scFv antibody
SEQ ID NO: 12
DIQMTQSPASLSASVGETVTITCRASENIYSFLAWYQQKQGKSPQLLVY
NTKTLTEGVPSRFSGSGSGTQFSLKINNLQPEDFGSYYCQHHYGIPLTF
GAGTKLELKGGGGSGGGGSGGGGSQVQLQQSGAELARPGASVKLSCKAS
GYTFTSYWMQWVKQRPGQGLEWIGAIYPGDGDTRYTQKFKGKATLTADK
SSSTAYMQLSNLTSEDSAVYYCARERTTGAPRYFDVWGAGTTVTVSS

Other exemplary anti-CD38 antibodies that may be used in the pharmaceutical compositions of the invention are those described in Int. Pat. Publ. No. WO05/103083, Intl. Pat. Publ. No. WO06/125640, Intl. Pat. Publ. No. WO07/042309, Intl. Pat. Publ. No. WO08/047242 or Intl. Pat. Publ. No. WO14/178820.

Anti-CD38 antibodies used in the methods of the invention may also be selected de novo from, e.g., a phage display library, where the phage is engineered to express human immunoglobulins or portions thereof such as Fabs, single chain antibodies (scFv), or unpaired or paired antibody variable regions (Knappik et al, J Mol Biol 296:57-86, 2000; Krebs et al, J Immunol Meth 254:67-84, 2001; Vaughan et al, Nature Biotechnology 14:309-314, 1996; Sheets et al, PITAS (USA) 95:6157-6162, 1998; Hoogenboom and Winter, J Mol Biol 227:381, 1991; Marks et al, J Mol Biol 222:581, 1991). CD38 binding variable domains may be isolated from e.g., phage display libraries expressing antibody heavy and light chain variable regions as fusion proteins with bacteriophage pIX coat protein as described in Shi et al, J. Mol. Biol. 397:385-96, 2010 and Intl. Pat. Publ. No. WO09/085462). The antibody libraries may be screened for binding to human CD38 extracellular domain, obtained positive clones further characterized, Fabs isolated from the clone lysates, and subsequently cloned as full length antibodies. Such phage display methods for isolating human antibodies are established in the art. See for example: U.S. Pat. Nos. 5,223,409; 5,403,484; and 5,571,698, 5,427,908, 5,580,717, 5,969,108, 6,172,197, 5,885,793; 6,521,404; 6,544,731; 6,555,313; 6,582,915; and 6,593,081.

### CD38 Depleting Antibodies

In some embodiments, the antibody suitable for depletion of CD38 cancer cells mediates antibody-dependent cell-mediated cytotoxicity.

As used herein the term “antibody-dependent cell-mediated cytotoxicity” or “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein, the term “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

As used herein, the term “effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody suitable for depletion of cancer cells is a full-length antibody. In some embodiments, the full-length antibody is an IgG1 antibody. In some embodiments, the full-length antibody is an IgG3 antibody.

In some embodiments, the antibody suitable for depletion of cancer cells comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of cancer cells is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP 1176195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian-like glycosylation pattern and capable of producing antibodies lacking fucose as glycosylation pattern (see for example EP1297172B1).

In some embodiments, the antibody suitable for depletion of cancer cells mediated complement dependant cytotoxicity.

As used herein, the term “complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody suitable for depletion of cancer cells mediates antibody-dependent phagocytosis.

As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

### CD38 Multispecific Antibodies:

In some embodiments, the antibody suitable for depletion of CD38 cancer cells is a multispecific antibody comprising a first antigen binding site directed against CD38 and at least one second antigen binding site directed against an effector cell as above described. In said embodiments, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the multispecific antibody is thus a bispecific antibody.

In some embodiments, the bispecific antibody is a BiTE. As used herein, the term “Bispecific T-cell engager” or “BiTE” refers to a bispecific antibody that is a recombinant protein construct composed of two flexibly connected single-chain antibodies (scFv). One of said scFv antibodies binds specifically to a selected, target cell-expressed tumour antigen (i.e. CD38), the second binds specifically to another molecule such as CD3, a subunit of the T-cell receptor complex on T cells. In some embodiments, the BiTE antibodies are capable of binding T cells transiently to target cells and, at the same time, activating the cytolytic activity of the T cells. The BiTE-mediated activation of the T cells requires neither specific T-cell receptors on the T cells, nor MHC I molecules, peptide antigens or co-stimulatory molecules on the target cell.

In some embodiments, the multispecific antibody of the present invention comprises the sequence as set forth in SED IQ NO:13.

>Sequence of Bi38-3
SEQ ID NO: 13
DIQMTQSPASLSASVGETVTITCRASENIYSFLAWYQQKQGKSPQLLVY
NTKTLTEGVPSRFSGSGSGTQFSLKINNLQPEDFGSYYCQHHYGIPLTF
GAGTKLELKGGGGSGGGGSGGGGSQVQLQQSGAELARPGASVKLSCKAS
GYTFTSYWMQWVKQRPGQGLEWIGAIYPGDGDTRYTQKFKGKATLTADK
SSSTAYMQLSNLTSEDSAVYYCARERTTGAPRYFDVWGAGTTVTVSSGG
GGSGGGGSGGGGSDIKLQQSGAELARPGASVKMSCKASGYTFTRYTMHW
VKQRPGQGLEWIGYINPSRGYTNYNQKFKDKATLTTDKSSSTAYMQLSS
LTSEDSAVYYCARYYDDHYCLDYWGQGTTLTVSSGGGGSGGGGSGGGGS
VDDIQLTQSPAIMSASPGEKVTMTCSASSSVSYMNWYQQKSGTSPKRWI
YDTSKLASGVPAHFRGSGSGTSYSLTISGMEAEDAATYYCQQWSSNPFT
FGSGTKLELKAAA

### CD38 Antibody-Drug Conjugates:

In some embodiments, the antibody suitable for depletion of cancer cells is conjugated to a therapeutic moiety, i.e. a drug.

In some embodiments, the therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of cancer cells is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of cancer cells is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (U.S. Pat. No. 5,663,149) and antifungal activity (Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of cancer cells is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al., Cancer Res 2010; 70(17): 6849-6858; Antonow D. et al., Cancer J 2008; 14(3): 154-169; Howard P. W. et al., Bioorg Med Chem Lett 2009; 19: 6463-6466 and Sagnou et al., Bioorg Med Chem Lett 2000; 10(18): 2083-2086.

In some embodiments, the antibody suitable for depletion of cancer cells is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody suitable for depletion of cancer cells is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of cancer cells is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

### CD38 CAR-T Cells

In some embodiments, the agent is a CAR-T cell wherein the CAR comprises at least an extracellular antigen binding domain specific for CD38.

In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the T cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28.

In some embodiments, the CAR comprises a chimeric fusion protein comprising an extracellular antigen binding domain specific for CD38, a transmembrane domain and an intracellular signaling domain comprising a functional signaling domain derived from a stimulatory molecule. In some embodiments, the CAR comprises a chimeric fusion protein comprising an extracellular antigen binding domain specific for CD38, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain specific for CD38, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain specific for CD38, a transmembrane domain and an intracellular signaling domain comprising at least two functional signaling domains derived from one or more costimulatory molecule(s) and a functional signaling domain derived from a stimulatory molecule.

In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

In particular aspects, CARs comprise fusions of single-chain variable fragments (scFv) derived from monoclonal antibodies that are specific for CD38, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

In some embodiments, the chimeric antigen receptor of the present invention comprises at least one VH and/or VL sequence of an antibody that is specific for CD38. In some embodiments, the portion of the CAR of the invention comprising an antibody or antibody fragment thereof that is specific for CD38 may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment specific for CD38. In a further aspect, the CAR comprises an antibody fragment that comprises a scFv that is specific for CD38.

In some embodiments, the CAR of the present invention consists of the amino acid sequence as set forth in SEQ ID NO:14 or SEQ ID NO:15.

SEQ ID NO: 14 > CAR CD38 1G
DIQMTQSPASLSASVGETVTITCRASENIYSFLAWYQQKOGKSPQLLVYNTKTLTEGVPSRFSGSGSGT
QFSLKINNLQPEDFGSYYCQHHYGIPLTFGAGTKLELKGGGGSGGGGSGGGGSQVOLQQSGAELARPGA
SVKLSCKASGYTFTSYWMQWVKQRPGQGLEWIGAIYPGDGDTRYTQKFKGKATLTADKSSSTAYMQLSN
LTSEDSAVYYCARERTTGAPRYFDVWGAGTTVTVSSLEHFVPVFLPAKPTTTPAPRPPTPAPTIASQPL
SLRPEACRPAAGGAVHTRGLDFACDIYIWAPLAGTCGVLLLSLVITLYCNHRNRVKFSRSADAPAYQQG
QNQLYNELNLGRREEYDVLDKRRGRDPEMGGKPQRRKNPQEGLYNELQKDKMAEAYSEIGMKGERRRGK
GHDGLYQGLSTATKDTYDALHMQALPPR
SEQ ID NO: 15 > CAR CD38 3G

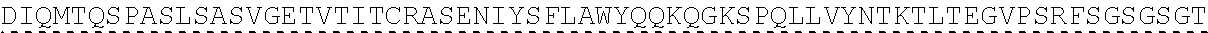

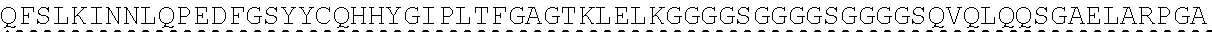

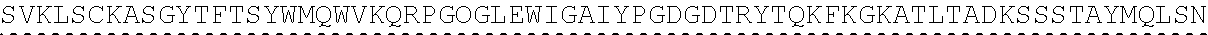

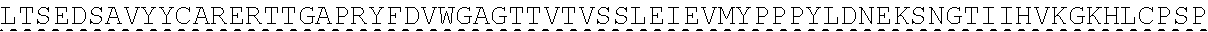

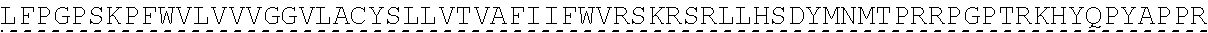

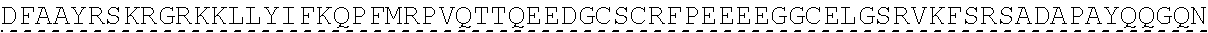

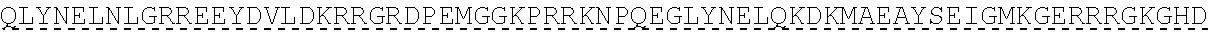

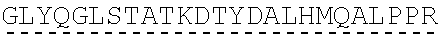

Methods for preparing CAR-T cells are well known in the art. In some embodiments, the cell (e.g., T cell) is transduced with a viral vector encoding a CAR. In some embodiments, the viral vector is a retroviral vector. In some embodiments, the viral vector is a lentiviral vector. In some embodiments, the cell may stably express the CAR. In some embodiments, the cell (e.g., T cell) is transfected with a nucleic acid, e.g., mRNA, cDNA, DNA, encoding a CAR. In some embodiments, the antigen binding domain of a CAR of the invention (e.g., a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In some embodiments, the chimeric antigen receptor of the present invention can be glycosylated, amidated, carboxylated, phosphorylated, esterified, N-acylated, cyclized via, e.g., a disulfide bridge, or converted into an acid addition salt and/or optionally dimerized or polymerized.

In some embodiments, the CAR activity can be controlled if desirable to optimize the safety and efficacy of a CAR therapy. There are many ways CAR activities can be regulated. For example, inducible apoptosis using, e.g., a caspase fused to a dimerization domain (see, e.g., Di et al., N Egnl. J. Med. 2011 Nov. 3; 365(18):1673-1683), can be used as a safety switch in the CAR therapy of the instant invention.

### Pharmaceutical Compositions:

Typically, the agent of the present invention is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the drug of the invention.

### Methods of Diagnosis:

A further object of the present invention relates to a method of diagnosing a T-cell lymphoma in a patient comprising detecting the expression level of CD38 in a sample obtained from the patient.

In some embodiments, the method of the present invention is particularly suitable for diagnosing a cutaneous T-cell lymphoma. More particularly, the method of the present invention is particularly suitable for diagnosing a Sézary syndrome.

As used herein, the term “sample” to any biological sample obtained from the purpose of evaluation in vitro. In some embodiments, the sample is sample is a blood sample. In some embodiments, the sample is PBMC sample. In some embodiments, the sample is a sample of (i) purified blood leukocytes, (ii) peripheral blood mononuclear cells or PBMC, (iii) purified lymphocytes, (iv) purified T cells, (v) purified CD4+ T cells or (vi) purified CD3+ T cells. In some embodiments, the biological sample is a tissue sample. The term “tissue sample” includes sections of tissues such as biopsy or autopsy samples and frozen sections taken for histological purposes. Thus in some embodiments, the tissue sample may result from a biopsy performed in the subject's skin. In some embodiments, the tissue sample may result from a biopsy performed in the subject's lymph node.

In some embodiments, the level of the marker is determined by immunohistochemistry (IHC). Immunohistochemistry typically includes the following steps i) fixing said tissue sample with formalin, ii) embedding said tissue sample in paraffin, iii) cutting said tissue sample into sections for staining, iv) incubating said sections with the binding partner specific for the marker, v) rinsing said sections, vi) incubating said section with a biotinylated secondary antibody and vii) revealing the antigen-antibody complex with avidin-biotin-peroxidase complex. Accordingly, the tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 125I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 300%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above descried), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In some embodiments, the level of the marker is determined by a flow-cytometric method. As used herein, the term “flow cytometric method” refers to a technique for counting cells of interest, by suspending them in a stream of fluid and passing them through an electronic detection apparatus. Flow cytometric methods allow simultaneous multiparametric analysis of the physical and/or chemical parameters of up to thousands of events per second, such as fluorescent parameters. Modern flow cytometric instruments usually have multiple lasers and fluorescence detectors. A common variation of flow cytometric techniques is to physically sort particles based on their properties, so as to purify or detect populations of interest, using “fluorescence-activated cell sorting”. As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest. Accordingly, FACS can be used with the methods described herein to isolate and detect the population of cells of the present invention. For example, fluorescence activated cell sorting (FACS) may be therefore used. involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. (Flowjo, Laluza . . . ), data input devices, e.g., interface ports, a mouse, a keyboard, etc., fluid handling components, power sources, etc. More particularly, the sample is contacted with a panel of antibodies specific for the specific market of the population of cells of the interest. Such antibodies or antigen-binding fragments are available commercially from vendors such as R&D Systems, BD Biosciences, e-Biosciences, Biolegend, Proimmune and Miltenyi, or can be raised against these cell-surface markers by methods known to those skilled in the art. In some embodiments, an agent that specifically bind to a cell-surface marker, such as an antibody or antigen-binding fragment, is labelled with a tag to facilitate the isolation and detection of population of cells of the interest. As used herein, the terms “label” or “tag” refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods to isolate and detect the cancer cells. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650. The aforementioned assays may involve the binding of the antibodies to a solid support. The solid surface could be a microtitration plate coated with the antibodies. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, California).

In some embodiments, the method further comprises detecting the expression level of a least one further marker. Typically, the marker is selected from the group consisting of KIR3DL2, PLS3, Twist and NKp46.

In the present specification, the name of each of the various markers of interest refers to the internationally recognised name of the corresponding gene, as found in internationally recognised gene sequences and protein sequences databases, including in the database from the HUGO Gene Nomenclature Committee that is available notably at the following Internet address: http://www.gene.ucl.ac.uk/nomenclature/index.html. In the present specification, the name of each of the various markers of interest may also refer to the internationally recognised name of the corresponding gene, as found in the internationally recognised gene sequences and protein sequences database Genbank. Through these internationally recognised sequence databases, the nucleic acid and the amino acid sequences corresponding to each of the marker of interest described herein may be retrieved by the one skilled in the art.

Multiplex tissue analysis techniques are particularly useful for quantifying several markers in the tissue sample. Such techniques should permit at least five, or at least ten or more biomarkers to be measured from a single tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, MD).

In some embodiments, the L-IHC method can be performed on any of a variety of tissue samples, whether fresh or preserved. The samples included core needle biopsies that were routinely fixed in 10% normal buffered formalin and processed in the pathology department. Standard five μιη thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10 μιη thick coated polymer backbone with 0.4 μιη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, MD). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, MD). That technique is described in U.S. Pat. No. 7,473,532.

In some embodiments, the method of the present invention utilized the multiplex IHC technique developed by GE Global Research (Niskayuna, NY). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image—processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the subject, ii) TMA samples are then stained with anti-antibodies having specificity of the protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the level of the marker is determined at nucleic acid level. Typically, the level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

In some embodiments, the method of the present invention further comprises comparing the expression level of the marker with a predetermined reference value wherein detecting a difference between the expression level of the marker and the predetermined reference value indicates whether the subject has a T-cell lymphoma.

In some embodiments, the predetermined reference value is a relative to a number or value derived from population studies, including without limitation, subjects of the same or similar age range, subjects in the same or similar ethnic group, and subjects having the same severity of lesion. Such predetermined reference values can be derived from statistical analyses and/or risk prediction data of populations obtained from mathematical algorithms and computed indices. In some embodiments, retrospective measurement of the level of the marker in properly banked historical subject samples may be used in establishing these predetermined reference values. Accordingly, in some embodiments, the predetermined reference value is a threshold value or a cut-off value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the marker in a group of reference, one can use algorithmic analysis for the statistic treatment of the measured levels of the marker in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

Typically, as demonstrated in EXAMPLE, the expression level of the CD38 is higher than the expression level determined in a sample from a healthy individual.

Monitoring the influence of agents (e.g., drug compounds) on the level of expression CD38 can be applied for monitoring the status of T-cell lymphoma in a patient with time. For example, the effectiveness of an agent to affect marker expression can be monitored during treatments of subjects receiving anti-T-cell lymphoma treatments.

Thus the present invention also provides a method for monitoring the effectiveness of treatment of a patient suffering from a T-cell lymphoma comprising the steps of:
(i) obtaining a pre-administration sample from a patient prior to administration of the agent;
(ii) detecting the level of expression of CD38 in the pre-administration sample;
(iii) obtaining one or more post-administration samples from the patient;
(iv) detecting the level of expression of the same marker(s) in the post-administration samples;
(v) comparing the level of expression of CD38 in the pre-administration sample with the level of expression of CD38 in the post-administration sample or samples; and
(vi) altering the administration of the agent to the patient accordingly.

For example, a worse diagnosis that is determined by assessing the expression level of CD38 during the course of treatment may indicate ineffective dosage and the desirability of increasing the dosage. Conversely, a better diagnosis that is determined by assessing the expression level of CD38 may indicate efficacious treatment and no need to change dosage.

Accordingly, the present invention also relates to a method for adapting an therapy in a patient suffering from a T-cell lymphoma, wherein said method comprises the steps of:
a) performing, on at least one sample collected from said patient, the in vitro diagnosis method that is disclosed herein; and
b) adapting the therapy of said patient by administering to said patient.

The invention also relates to a kit for performing the diagnosis methods as described above. The kit comprises a plurality of reagents, in particular at least one agent that is capable of binding specifically to the CD38 marker. Suitable reagents for binding with a marker protein include antibodies, antibody derivatives, antibody fragments, and the like. Suitable reagents for binding with a marker nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like. The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of the in vitro diagnosis method of the invention, and the like.

### Methods for Predicting the Survival Time of a Patient:

Another aspect of the invention relates to a method for predicting the survival time of a patient suffering from a T-cell lymphoma comprising i) determining the expression level of CD38 in a sample obtained from the patient ii) comparing the expression level determined at step i) with a predetermined reference value and iii) providing a good prognosis when the expression level determined at step i) is lower than its predetermined reference value, or providing a bad prognosis when the expression level determined at step i) is higher than its predetermined reference value.

As used herein, the term “survival time” denotes the percentage of people in a study or treatment group who are still alive for a certain period of time after they were diagnosed with or started treatment for a disease, such as a T-cell lymphoma (according to the invention). The survival time rate is often stated as a five-year survival rate, which is the percentage of people in a study or treatment group who are alive five years after their diagnosis or the start of treatment. As used herein and according to the invention, the term “survival time” can regroup the term “Overall survival (OS)”.

As used herein, the term “OS” denotes the time from diagnosis of a disease such as a T-cell lymphoma (according to the invention) until death from any cause. The overall survival rate is often stated as a two-year survival rate, which is the percentage of people in a study or treatment group who are alive two years after their diagnosis or the start of treatment.

Measuring the expression level of CD38 can be done by measuring the gene expression level of CD38 or by measuring the level of the protein CD38 and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence or mRNA) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrromethene boron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT® (obtained, for example, from Life Technologies (Quantum Dot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the band gap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlsbad, Calif.). Additional labels include, for example, radioisotopes (such as ³H), metal chelates such as DOTA and DPTA chelates of radioactive or paramagnetic metal ions like Gd3+.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase. Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH). Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am.1. Pathol. 157:1467-1472, 2000 and U.S. Pat. No. 6,942,970. Additional detection methods are provided in U.S. Pat. No. 6,280,929. Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP (dinitrophenol), and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153. It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 nm) and a second specific binding agent (in this case an anti-DNP antibody, or antibody fragment, labelled with a second fluorophore (for example, a second spectrally distinct QUANTUM DOT®, e.g., that emits at 705 nm). Additional probes/binding agent pairs can he added to the multiplex detection scheme using other spectrally distinct fluorophores. Numerous variations of direct, and indirect (one step, two step or more) can be envisioned, all of which are suitable in the context of the disclosed probes and assays. Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TFRC, GAPDH, GUSB, TBP and ABL1. This normalization allows the comparison of the expression level in one sample, e.g., a patient sample, to another sample, or between samples from different sources.

According to the invention, the level of CD38 proteins may also be measured and can be performed by a variety of techniques well known in the art. For measuring the expression level of CD38, techniques like ELISA (see below) or ELLA allowing to measure the level of the soluble proteins are particularly suitable. In the present application, the “level of protein” or the “protein level expression” or the “protein concentration” means the quantity or concentration of said protein. In another embodiment, the “level of protein” means the level of CD38 protein fragments. In still another embodiment, the “level of protein” means the quantitative measurement of CD38 protein expression relative to an internal control.

Typically protein concentration may be measured for example by capillary electrophoresis-mass spectroscopy technique (CE-MS) or ELISA performed on the sample. Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS), etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like. More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Methods of the invention may comprise a step consisting in comparing the proteins and fragments concentration with a control value. As used herein, “concentration of protein” refers to an amount or a concentration of a transcription product, for instance the protein CD38. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the protein CD38. Thus, in a particular embodiment, fragments of CD38 protein may also be measured.

Predetermined reference values used for comparison of the expression levels may comprise “cut-off” or “threshold” values that may be determined as described herein. Each reference (“cut-off”) value for CD38 level may be predetermined by carrying out a method comprising the steps of:
a) providing a collection of samples from patients suffering of a cancer and/or samples of the corresponding uninvolved tissues as described in the invention;
b) determining the level of CD38 for each sample contained in the collection provided at step a);
c) ranking the tumor tissue samples according to said level
d) classifying said samples in pairs of subsets of increasing, respectively decreasing, number of members ranked according to their expression level,
e) providing, for each sample provided at step a), information relating to the actual clinical outcome for the corresponding cancer patient;
f) for each pair of subsets of samples, obtaining a Kaplan Meier percentage of survival curve;
g) for each pair of subsets of samples calculating the statistical significance (p value) between both subsets
h) selecting as reference value for the level, the value of level for which the p value is the smallest.

For example the expression level of CD38 may be assessed for 100 cancer samples of 100 patients. The 100 samples are ranked according to their expression level. Sample 1 has the highest expression level and sample 100 has the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated. The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels. In routine work, the reference value (cut-off value) may be used in the present method to discriminate cancer samples and therefore the corresponding patients.

Kaplan-Meier curves of percentage of survival as a function of time are commonly used to measure the fraction of patients living for a certain amount of time after treatment and are well known by the man skilled in the art. The man skilled in the art also understands that the same technique of assessment of the expression level of a protein should of course be used for obtaining the reference value and thereafter for assessment of the expression level of a protein of a patient subjected to the method of the invention.

A further object of the invention relates to kits for performing the methods of the invention, wherein said kits comprise means for measuring the expression level of CD38 in the sample obtained from the patient. The kits may include probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively, the kit of the invention may comprise amplification primers that may be pre-labelled or may contain an affinity purification or attachment moiety. The kit may further comprise amplification reagents and also other suitably packaged reagents and materials needed for the particular amplification protocol.

The invention will be further illustrated by the following figures and examples. However, these examples and figures should not be interpreted in any way as limiting the scope of the present invention.

### FIGURES

FIG. 1. CD38 expression of T-cell lymphoma cell lines. Cytometry dot plots of CD38 expression on Seax, Myla and HUT78 cutaneous T-cell lymphoma cell lines (white) versus control isotype (black), showing significant CD38 expression by the Seax and HUT78 cell lines.

FIG. 2. CD38 expression in fresh peripheral blood tumor cells from patients with Sézary syndrome. A—Flow cytometry dot plots of CD38 expression on CD4+ blood cells of 5 patients with Sézary syndrome, showing significant expression of CD38 by CD4+ peripheral blood cells in 3 of them. B—Flow cytometry dot plots and histograms of CD38 expression in gated peripheral blood CD3 T cells (dot plots) and CD3+CD4+KIR3DL2+ blood Sézary cells (blue histograms) versus control isotype (red histograms) in 4 patients with Sézary syndrome.

FIG. 3. Expression of CD38 by blood tumor cells in Sézary syndrome and in vitro depleting efficacy of the anti-human CD38 monoclonal antibody isatuximab. A—Gating strategy to study CD38 expression by flow cytometry in CD4+ T cells of the 18 Sézary patients. Sézary cells (SCs) were identified as CD4+/low KIR3DL2+ or clonal TCR-Vb lymphocytes displaying monophasic TRBC1 expression. Benign CD4+ Tcells were non SCs with a biphasic TRBC1 expression. B—Patterns of CD38 expression by HD's derived CD8+(blue) and CD4+ T cells (green) (left). Details of CD38 expression by benign CD4+ T cells and SCs from 3 patients with Sézary syndrome from the total cohort of 18 patients studied by flow cytometry. C—CD38 Mean Fluorescence Intensity (MFI, left panel) and % (right panel) of CD38int and CD38hi cells within Sézary cells (SCs), Benign CD4+ T cells and HD's CD4 and CD8 T-cells. Medians were compared using Mann Whitney U test, p<0.05 considered significant, **** p<0.0001. D—Antibody-dependent cell phagocytosis (ADCP) using control Ig (Igc), anti-human CD38 monoclonal antibodies isatuximab and alemtuzumab on four Sézary patient's cells using THP-1 cells as effectors. Effectors and targets were at 1-1 ratio and incubated for 4 h with antibodies at 1 ug/ml. ADCP was evaluated by the percentage of CD32+ CFSE+ cells. Medians and interquartile ranges have been indicated and p-value from ANOVA comparisons. *p<0.05. Correlation between CD38 mean fluorescence intensity (MFI) on Sézary target cells (MFI CD38/control isotype) and ADCP efficacy is shown in the lower panel (Spearman correlation, p=0.08). E—Antibody-dependent cell cytotoxicity (ADCC) using control human Ig (hIgc), anti-human CD38 monoclonal antibody isatuximab on three Sézary patient's cells using autologous NK cells as effectors at various effector/target (E-T) ratios. ADCC was evaluated by the percentage of KIR3DL2+ apoptotic (viability dye, VD+) cells. Medians and interquartile ranges have been indicated and p-value from ANOVA comparison. *p<0.05.

FIG. 4. Expression of CD38 in skin and lymph node of cutaneous T-cell lymphomas. A-Details of CD38 expression by single cell RNA sequencing data in skin of one patient with aggressive epidermotropic cutaneous T-cell lymphoma (AECTCL), 2 patients with stage IIB mycosis fungoides (MF), one patient with stage IIIA MF, 1 patient with Sézary syndrome (SS) and 4 healthy donors. B—Overall survival according to the presence or not of CD38 expression by immunohistochemistry in 51 patients with CTCL, immunohistochemistry and follow-up data. Overall survival probability was estimated from the time of the diagnostic biopsy to last follow-up or death from any cause. The effect of CD38 expression was analyzed by univariate Cox analysis.

### EXAMPLE 1

### Methods

### CD38 Expression of T-Cell Lymphoma Cell Lines

Cells were incubated with control isotype or anti-CD38 antibody (clone HIT-2) during 15 min at 4° C., then washed in PBS and analyzed on a LSRX20 flow cytometer

CD38 Expression in Fresh Peripheral Blood Tumor Cells from Patients with Sézary Syndrome

Study of CD38 expression by flow cytometry on peripheral blood mononuclear cells of 5 patients with Sézary syndrome using anti-CD4, CD158k (=KIR3DL2, surface marker of Sézary cells), and CD38 antibodies or control isotype after information and signature of informed consent.

### Results

### CD38 Expression of T-Cell Lymphoma Cell Lines

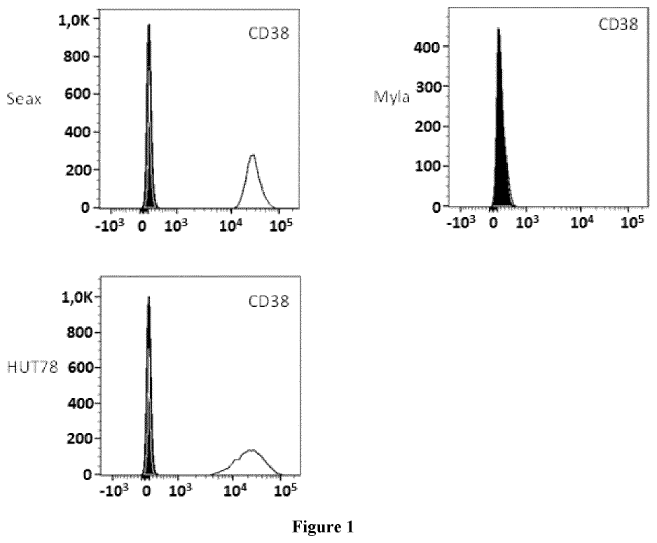

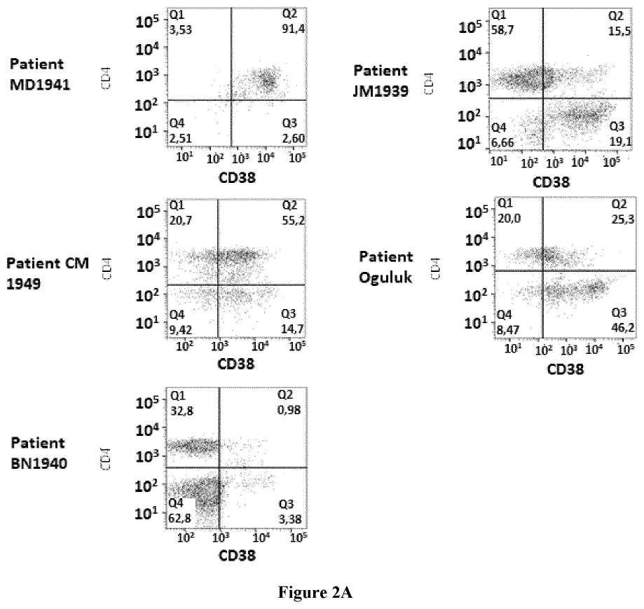

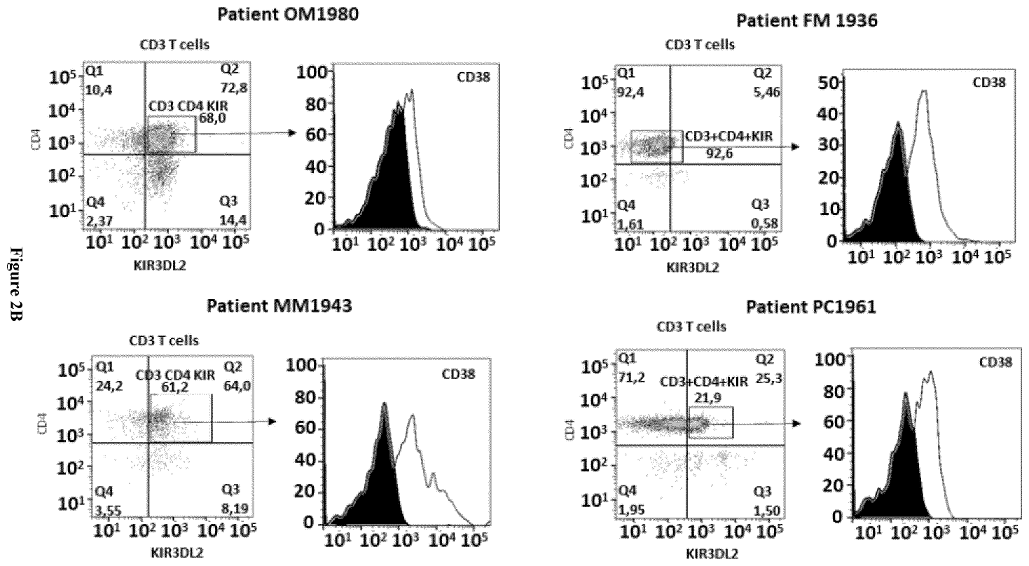

Seax, Myla and HuT78 Sézary syndrome cell lines were stained with anti-CD38 antibody or control isotype and CD387 expression was analyzed by flow cytometry (FIG. 1).

CD38 Expression in Fresh Peripheral Blood Tumor Cells from Patients with Sézary Syndrome

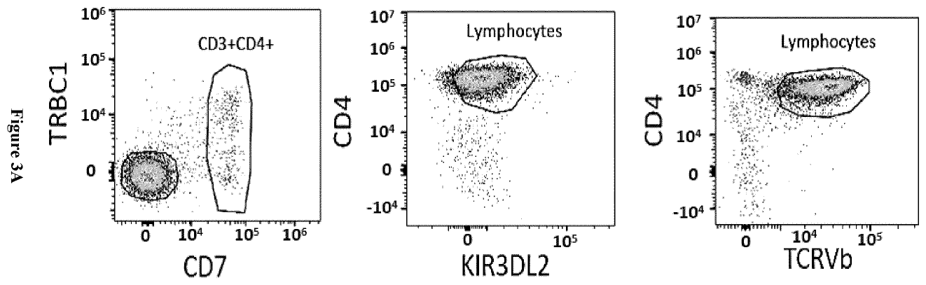

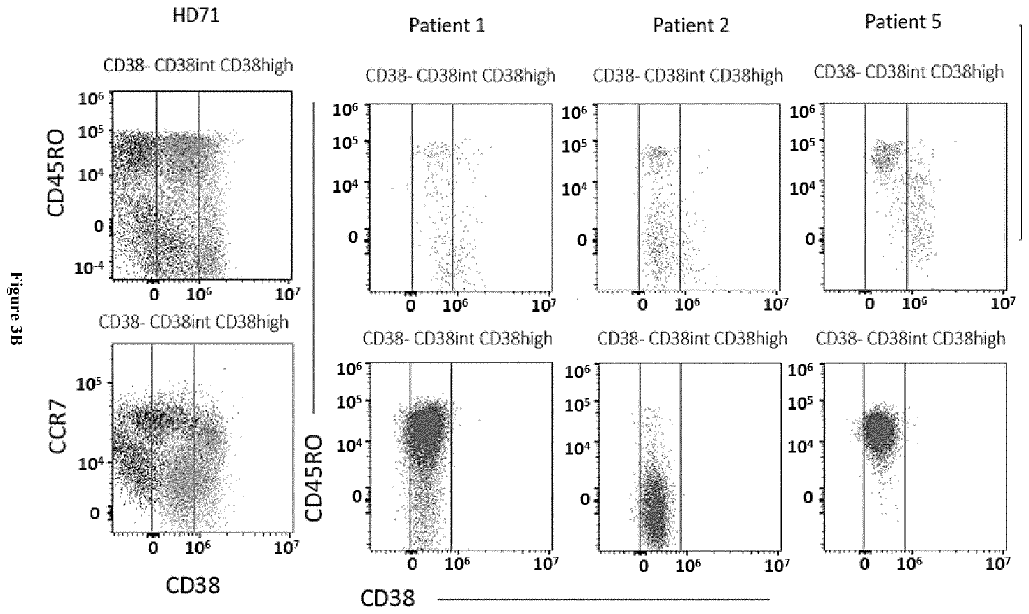

We show significant expression of CD38 by CD4+ peripheral blood cells in 3 patients (FIG. 2A). Overexpression of CD38 by circulating CD4+KIR3DL2+ tumor cells from patients with Sézary syndrome compared to reactive KIR3DL2− CD4 T cells (FIG. 2B). Four different Sézary patient's cells were stained with anti-CD4, anti-KIR3DL2 and anti-CD38 antibodies. The CD198 expression was analyzed on the CD4+KIR3DL2+ tumor cell population.

### Conclusions:

This study of the regulatory T phenotype of Sézary cells led to the discovery of the expression of CD38 by Sézary cells. CD38 therefore appears as a useful diagnostic, prognostic and follow-up marker, and as a potential therapeutic target in T-cell lymphomas. Therapeutic depletion of CD38-expressing cells could eliminate tumor cells and also activate the anti-tumor immunity in T-cell lymphomas.

### EXAMPLE 2

The treatment of advanced-stage cutaneous T-cell lymphomas (CTCL) remains challenging. CTCL course is characterized by multiple relapses; the disease may escape from therapeutic monoclonal antibodies by different mechanisms including target loss (11-13). Here we combined immunohistochemistry, multiparameter flow cytometry, and single cell RNA sequencing to characterize the expression of CD38 in skin, blood and lymph nodes in a total of 67 CTCL cases.

### Methods

### Patients

Adult CTCL patients were included after informed consent. CTCL staging was defined according to the international criteria (14). This study received the agreement of the local ethics committee (CPP2019-A01158-49) and was conducted in accordance with the principles of the Helsinki declaration.

### Immunohistochemistry

FFPE tissue sections from 56 samples (52 patients) were immunostained using anti-CD3 (polyclonal, Dako, Glostrup, Denmark), CD4 (SP35 clone, Roche, Basel, Switzerland), CD7 (CBC.37 clone, Dako), CD8 (C8/144B clone, Dako), CD30 (Ber-H2 clone, Dako), CD38 (SP149) antibodies and analyzed on a BenchMark ULTRA automated immunostainer (Roche).

### Flow Cytometry

The Sézary cell lines HuT-78 and SeAx were analyzed by flow cytometry using anti-CD38 antibody (HIT2 clone) or control isotype. Peripheral blood mononuclear cells (PBMCs) from 18 Sézary patients and 10 age-matched healthy donors (HD) were analyzed using a 34-color panel; acquisition was performed on Cytek® Aurora cytometer. Sézary cells (SCs) were identified as CD4+/low KIR3DL2+ or clonal TCRVb lymphocytes displaying monophasic TRBC1 expression. Benign CD4+ T-cells were characterized using a subtractive gating strategy, excluding SCs and showing a biphasic TRBC1 expression pattern, with both discrete positive and negative subsets.

### Antibody-Dependent Cell Phagocytosis (ADCP)

THP-1 cells were mixed with CFSE-stained patient cells (80% tumor cells in T-cell gate) at 1-1 ratio and incubated for 4 h with antibodies at 1 g/ml. ADCP was evaluated by the % CD32+ CFSE+ cells.

### Antibody-Dependent Cell Cytotoxicity (ADCC)

PBMC were mixed with patient's cells and incubated for 16 h with antibodies at 10 g/ml. ADCC was evaluated by the percentage of KIR3DL2+ apoptotic (viability dye, VD+) cells.

### Single-Cell RNA Sequencing Analysis

Publicly available data were accessed from the Gene Expression Omnibus (GEO) database (accession GSE128531). This contains gene expression profiles of skin biopsies from four healthy volunteers, two tumor-stage MF patients, one aggressive epidermotropic CTCL, one erythrodermic MF patient and one Sézary patient as described in (15). Biopsy proceeding, library preparation, single-cell RNA-sequencing and patients characteristics are described in (15). Briefly, droplet-based sequencing was performed after enzymatic digestion of the tissue samples. A median of 4467 cells (range, 2200-9272) were analyzed per sample.

### Results

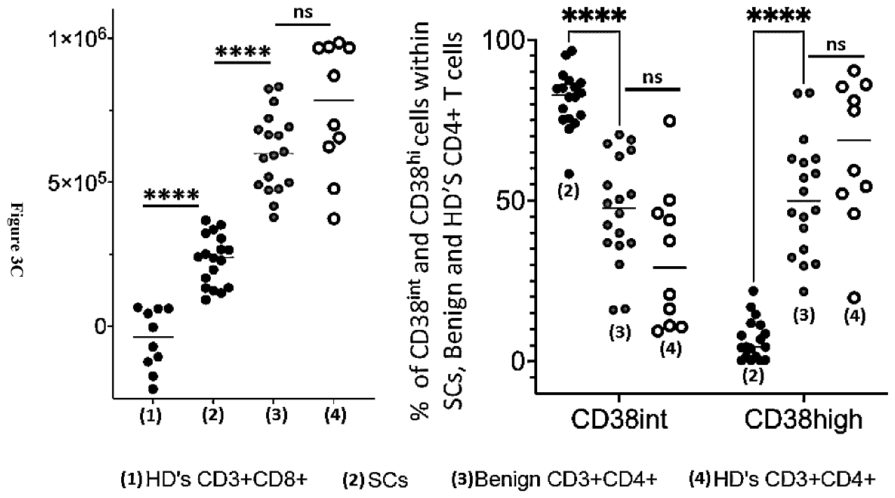

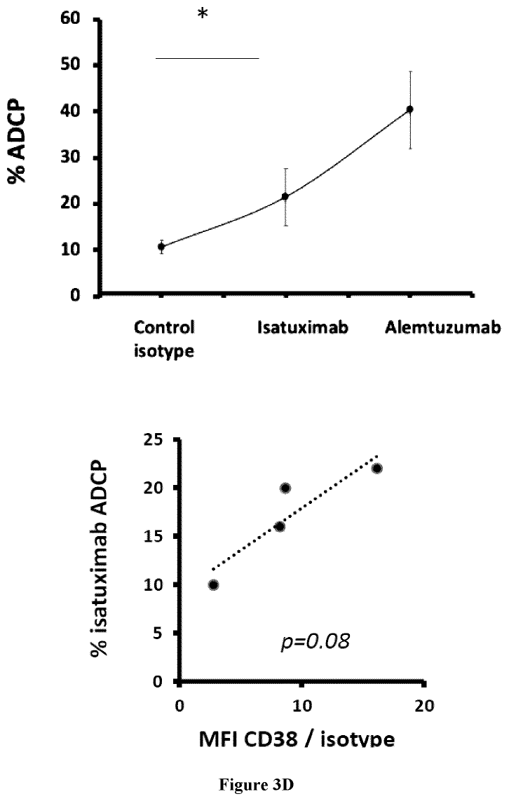

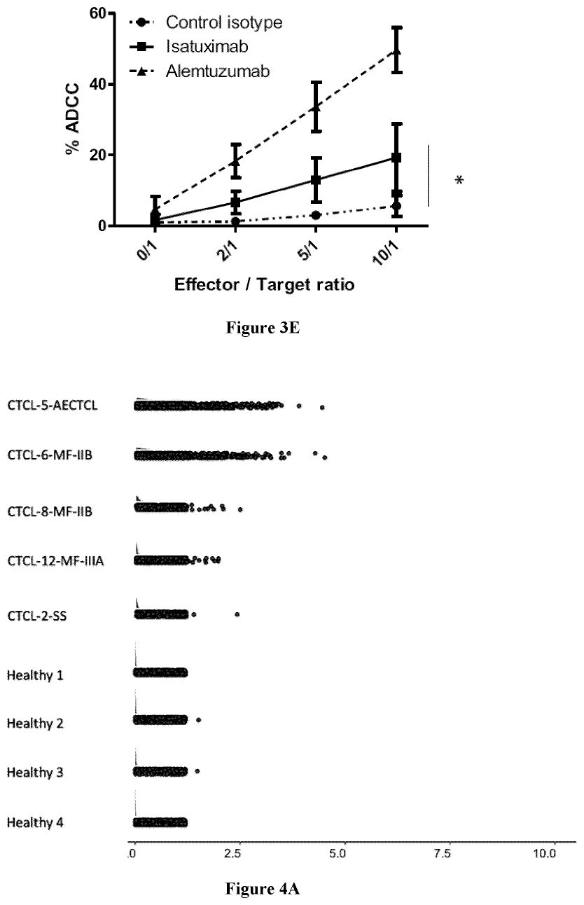

CD38 was uniformly expressed by the Sézary cell lines SeAx and HuT78 (FIG. 1 and FIG. 3A). CD38 was found predominantly expressed by CD45RO-CCR7+naïve cells from HD (FIG. 3B and data not shown). Detailed flow cytometry analysis of SCs and T-cells in 18 patients and 10 HD showed that SCs were unequivocally CD38int, benign and HD's derived CD4 T-cells were CD38int and CD38hi, and CD8 T-cells from HDs were CD38-negative/CD38int (FIG. 3C and data not shown). Malignant and benign CD4+CD38+ subsets segregated into separate clusters, according to their respective expression of chemokine and immune checkpoint receptors (data not shown).

Three Sézary patients with refractory disease that had escaped to mogamulizumab and lacutamab displayed CD38 expression on SCs. The data illustrates the case of Patient 6 with persistent CD38 expression on blood SCs after mogamulizumab treatment, while CCR4 and PD1 expression were lost after the disease had escaped.

Isatuximab, an anti-CD38 antibody, induced ADCP, as measured by CD32 expression by THP-1 cells cultured with peripheral blood SCs from four Sézary patients in comparison to control isotype and alemtuzumab (anti-CD52) (positive control) (FIG. 3D). A nonstatistically significant correlation between CD38 expression (MFI CD38/isotype) on target cells and % isatuximab-induced ADCP was observed (Spearman, p=0.08) probably due to the limited number of patients (FIG. 3D). ADCC was observed ex vivo on three Sézary patients' cells using isatuximab (anti-CD38) versus control isotype, or alemtuzumab (FIG. 3E).

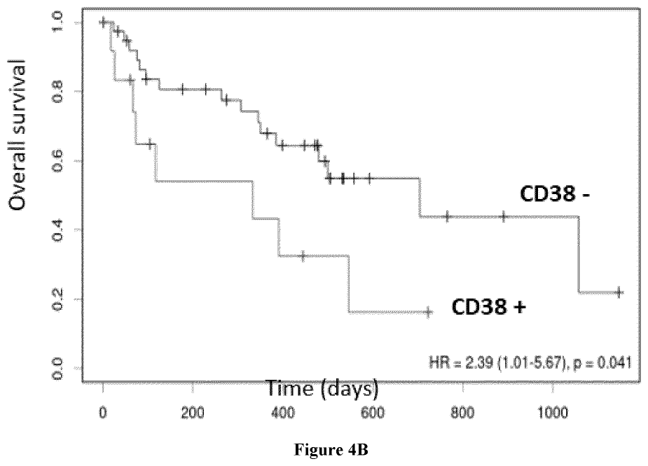

To better characterize CD38 expression by skin cell subsets in CTCL, single-cell RNA sequencing data of CTCL patients' skin were analyzed and confirmed CD38 expression in CTCL lesional skin. A higher number of CD38hi skin cells was found in CTCL skin samples compared to healthy donors (p=0.02, FIG. 4A and Table 1). CD38 RNA was expressed by the skin lymphocyte subset in CTCL patients, and to a lesser extent by macrophages (data not shown). Within the skin lymphocyte subset, CD38 was coexpressed with CTCL tumor cell markers (KIR3DL2, TOX) and with different markers identified as specific CTCL cell markers on this population by Gaydosik et al. (ACTG1, HMGN1, NUSAP1, STMN1 and others). CD38 was also coexpressed with cytotoxic cell markers (CCL5, GZMA) (data not shown). Of note, transformed cells in MF frequently express cytotoxic markers (16). This suggests that CD38 is expressed by skin tumor cells in advanced CTCL.

CD38 expression was confirmed in skin or lymph node of CTCL patients by immunohistochemistry in 12 out of 56 tested skin and lymph node samples from 52 different CTCL patients (3 of these patients were also studied by blood flow cytometry (Table 2 and data not shown)). CD38 expression was mostly found in SS with large-cell transformation (6/14 samples), primary cutaneous gamma delta T-cell lymphoma (1/2), primary cutaneous NK/T cell lymphoma (2/2) thus suggesting that CD38 expression in skin was associated with aggressive CTCL subtypes. The percentages of expression varied from 5 to 70%. Two transformed SS cases and 1 non-transformed SS had CD38 expression in involved lymph node (N3 ISCL/EORTC stage) (data not shown). The overall survival was compared between CTCL patients displaying CD38 positivity by immunohistochemistry and patients without detected CD38 expression. CD38 expression in CTCL altogether was associated with significantly shorter overall survival (hazard ratio, 2.39, 95% CI 1.01-5.67, p=0.041) (FIG. 4B). Finally, we observed a clinical response in skin of a Sézary patient treated with daratumumab for concomitant multiple myeloma (data not shown).

TABLE 1
Numbers and % of cells

| | Total | CD38+ | % of | CD38++ | % of |
|---|---|---|---|---|---|
| | number | number | cells | number | cells |
| Diagnosis | of cells | of cells | CD38+ | of cells | CD38++ |
| Healthy 1 | 3805 | 4 | 0.11 | 0 | 0 |
| Healthy 2 | 2200 | 3 | 0.14 | 1 | 0.05 |
| Healthy 3 | 4847 | 8 | 0.17 | 0 | 0 |
| Healthy 4 | 3327 | 2 | 0.06 | 0 | 0 |
| CTCL-5-AECTCL | 9272 | 1024 | 11.0 | 4.35 | 403 |
| CTCL-2-SS | 4467 | 6 | 0.13 | 1 | 0.02 |
| CTCL-6-MF IIB 1 | 4730 | 503 | 10.6 | 168 | 3.55 |
| CTCL-8-MF IIB 2 | 8587 | 54 | 0.63 | 4 | 0.05 |
| CTCL-12-MF IIIA | 3607 | 49 | 1.36 | 9 | 0.25 |

| | |
|---|---|
| CTCL vs. healthy | Two-tailed P value for number of CD38++ cells |
| | using Mann-Whitney U-test: p = 0.02 |

TABLE 2
Immunohistochemistry data

| | | Lymph | % positive | Staining |
|---|---|---|---|---|
| Disease | Skin | node | cells | intensity |
| MF | 0/6 | | | |
| MF with large-cell | 1/8 | 0/1 | 10 | + |
| transformation | | | | |
| SS | 0/11 | 1/3 | 70 | ++ |
| SS with large-cell | 4/11 | 2/3 | 5, 10, 20 and | + in 3 cases |
| transformation | | | 25 (skin); 5 | and ++ in 1 |
| | | | and 10 (node) | (skin); + in |
| | | | | 2 cases (node) |
| CD30+ | 1/4 | | 20 | + |
| pcALCL | | | | |
| pcGDTCL | 1/2 | | 20 | + |
| pcNKTCL | 2/2 | | 30 and 70 | + and ++ |
| pcPTCL- NOS | 1/5 | | 5 | ++ |

Abbreviations: MF, mycosis fungoides; LCT, large-cell transformation; SS, Sézary syndrome; pcALCL, primary cutaneous anaplastic large-cell lymphoma; NKTCL, NK/T cell lymphoma; PTCL-NOS, primary cutaneous peripheral T-cell lymphoma, not otherwise specified.

### REFERENCES

Throughout this application, various references describe the state of the art to which this invention pertains. The disclosures of these references are hereby incorporated by reference into the present disclosure.
1. Willemze R, Cerroni L, Kempf W, Berti E, Facchetti F, Swerdlow S H, et al. The 2018 update of the WHO-EORTC classification for primary cutaneous lymphomas. Blood. 18 avr 2019; 133(16):1703-14.
2. Hurabielle C, Thonnart N, Ram-Wolff C, Sicard H, Bensussan A, Bagot M, et al. Usefulness of KIR3DL2 to Diagnose, Follow-Up, and Manage the Treatment of Patients with Sézary Syndrome. Clin Cancer Res Off J Am Assoc Cancer Res. 15 juill 2017; 23(14):3619-27.
3. Olsen E A, Whittaker S, Kim Y H, Duvic M, Prince H M, Lessin S R, et al. Clinical end points and response criteria in mycosis fungoides and Sézary syndrome: a consensus statement of the International Society for Cutaneous Lymphomas, the United States Cutaneous Lymphoma Consortium, and the Cutaneous Lymphoma Task Force of the European Organisation for Research and Treatment of Cancer. J Clin Oncol Off J Am Soc Clin Oncol. 20 juin 2011; 29(18):2598-607.
4. Roelens M, de Masson A, Ram-Wolff C, Maki G, Cayuela J-M, Marie-Cardine A, et al. Revisiting the initial diagnosis and blood staging of mycosis fungoides and Sézary syndrome with the KIR3DL2 marker. Br J Dermatol. juin 2020; 182(6):1415-22.
5. Bagot M, Porcu P, Marie-Cardine A, Battistella M, William B M, Vermeer M, et al. IPH4102, a first-in-class anti-KIR3DL2 monoclonal antibody, in patients with relapsed or refractory cutaneous T-cell lymphoma: an international, first-in-human, open-label, phase 1 trial. Lancet Oncol. août 2019; 20(8):1160-70.
6. Kim Y H, Bagot M, Pinter-Brown L, Rook A H, Porcu P, Horwitz S M, et al. Mogamulizumab versus vorinostat in previously treated cutaneous T-cell lymphoma (MAVORIC): an international, open-label, randomised, controlled phase 3 trial. Lancet Oncol. sept 2018; 19(9):1192-204.
7. Bonnet P, Battistella M, Roelens M, Ram-Wolff C, Herms F, Frumholtz L, et al. Association of autoimmunity and long-term complete remission in patients with Sézary syndrome treated with mogamulizumab. Br J Dermatol. févr 2019; 180(2):419-20.
8. Khodadoust M S, Rook A H, Porcu P, Foss F, Moskowitz A J, Shustov A, et al. Pembrolizumab in Relapsed and Refractory Mycosis Fungoides and Sézary Syndrome: A Multicenter Phase I I Study. J Clin Oncol Off J Am Soc Clin Oncol. 1 janv 2020; 38(1):20-8.
9. Bensussan A, Janela B, Thonnart N, Bagot M, Musette P, Ginhoux F, et al. Identification of CD39 as a Marker for the Circulating Malignant T-Cell Clone of Sézary Syndrome Patients. J Invest Dermatol. mars 2019; 139(3):725-8.
10. Jariwala N, Benoit B, Kossenkov A V, Oetjen L K, Whelan™, Cornejo C M, et al. TIGIT and Helios Are Highly Expressed on CD4+ T Cells in Sézary Syndrome Patients. J Invest Dermatol. janv 2017; 137(1):257-60.
11. Roelens M, de Masson A, Andrillon A, Ram-Wolff C, Biard L, Boisson M, et al. Mogamulizumab induces long term immune restoration and reshapes tumor heterogeneity in Sézary syndrome. Br J Dermatol. 2022; 186(6):1010-1025.
12. de Masson A, Darbord D, Dobos G, Boisson M, Roelens M, Ram-Wolff C, et al. Macrophage-derived CXCL9 and CXCL11, T-cell skin homing, and disease control in mogamulizumab-treated CTCL patients. Blood. 2022; 139(12):1820-1832.
13. Beygi S, Duran G E, Fernandez-Pol S, Rook A H, Kim Y H, Khodadoust M S. Resistance to mogamulizumab is associated with loss of CCR4 in cutaneous T-cell lymphoma. Blood. 2022; 139(26):3732-6.
14. Olsen E A, Whittaker S, Willemze R, Pinter-Brown L, Foss F M, Geskin L J, et al. Primary Cutaneous Lymphoma: Recommendations for Clinical Trial Design and Staging Update from the ISCL, USCLC, and EORTC. Blood. 2021; blood.2021012057.
15. Gaydosik A M, Tabib T, Geskin L J, Bayan C A, Conway J F, Lafyatis R, et al. Single-Cell Lymphocyte Heterogeneity in Advanced Cutaneous T-cell Lymphoma Skin Tumors. Clin Cancer Res Off J Am Assoc Cancer Res. 2019; 25(14):4443-54.
16. Vermeer M H, Geelen F A, Kummer J A, Meijer C J, Willemze R. Expression of cytotoxic proteins by neoplastic T cells in mycosis fungoides increases with progression from plaque stage to tumor stage disease. Am J Pathol. 1999; 154(4):1203-10.

1. A method of treating a T-cell lymphoma in patient in need thereof comprising administering to the patient a therapeutically effective amount of an agent capable of inducing cell death of CD38 expressing cancer cells.

2. The method of claim 1 wherein the T-cell lymphoma is cutaneous T-cell lymphoma.

3. The method of claim 2 wherein the T-cell lymphoma is Sézary syndrome.

4. The method of claim 1 wherein the agent is an antibody having binding affinity for CD38.

5. The method of claim 4 wherein the agent is an antibody directed against a least one extracellular domain of CD38 and leads to the depletion of CD38 expressing cancer cells.

6. The method of claim 4 wherein the antibody having binding affinity for CD38 comprises:
a heavy chain comprising i) the H-CDR1 as set forth in SEQ ID NO:4, ii) the H-CDR2 as set forth in SEQ ID NO:5 and iii) the H-CDR3 as set forth in SEQ ID NO:6, and,
a light chain comprising i) the L-CDR1 as set forth in SEQ ID NO:7, ii) the L-CDR2 as set forth in SEQ ID NO:8 and iii) the L-CDR3 as set forth in SEQ ID NO:9.

7. The method of claim 4 wherein the antibody depletes CD38 cancer cells and mediate antibody-dependent cell-mediated cytotoxicity.

8. The method of claim 4 wherein the antibody is a multispecific antibody comprising a first antigen binding site directed against CD38 and at least one second antigen binding site directed against an effector cell.

9. The method of claim 8 wherein the multispecific antibody comprises the sequence as set forth in SED ID NO:13.

10. The method of claim 5 wherein the antibody is conjugated to a cytotoxic moiety.

11. The method of claim 1 wherein the agent is a CAR-T cell wherein the CAR comprises at least an extracellular antigen binding domain specific for CD38.

12. The method of claim 11 wherein the CAR consists of the amino acid sequence as set forth in SEQ ID NO:14 or SEQ ID NO:15.

13. A method of diagnosing a T-cell lymphoma in a patient comprising detecting an expression level of CD38 in a sample obtained from the patient and comparing the expression level of the CD38 with a predetermined reference value, wherein detecting a difference between the expression level of the CD38 and the predetermined reference value indicates that the subject has a T-cell lymphoma.

14. The method of claim 13, wherein the T-cell lymphoma is a cutaneous T-cell lymphoma.

15. The method of claim 14, wherein the cutaneous T-cell lymphoma is a Sézary syndrome lymphoma.

16. The method of claim 13 further comprising detecting an expression level of at least one further marker selected from the group consisting of KIR3DL2, PLS3, Twist and NKp46.

17. A method for predicting the survival time of a patient suffering from a T-cell lymphoma comprising i) determining an expression level of CD38 in a sample obtained from the patient ii) comparing the expression level determined at step i) with a predetermined reference value and iii) providing a good prognosis when the expression level determined at step i) is lower than its predetermined reference value, or providing a bad prognosis when the expression level determined at step i) is higher than its predetermined reference value.


SEQUENCE LISTING (15 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 8 aa, synthetic construct
GYTFTSYW

SEQ ID NO: 5 - protein, 8 aa, synthetic construct
IYPGDGDT

SEQ ID NO: 6 - protein, 14 aa, synthetic construct
ARERTTGAPRYFDV

SEQ ID NO: 7 - protein, 6 aa, synthetic construct
ENIYSF

SEQ ID NO: 8 - skipped

SEQ ID NO: 9 - protein, 9 aa, synthetic construct
QHHYGIPLT

SEQ ID NO: 10 - protein, 121 aa, synthetic construct
QVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSS

SEQ ID NO: 11 - protein, 107 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELK

SEQ ID NO: 12 - protein, 243 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELKGGGGSGGGGSGGGGSQVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSS

SEQ ID NO: 13 - protein, 503 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELKGGGGSGGGGSGGGGSQVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSSGGGGSGGGGSGGGGSDIKLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSVDDIQLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLELKAAA

SEQ ID NO: 14 - protein, 442 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELKGGGGSGGGGSGGGGSQVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSSLEHFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 508 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELKGGGGSGGGGSGGGGSQVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSSLEIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR